(19) United States
(12) Patent Application Publication
(10) Pub. No.: US 2018/0008474 A1
(43) Pub. Date: Jan. 11, 2018

(54) BANDAGE HAVING HEMOSTATIC GAUZE AND METHOD THEREFOR

(71) Applicant: JONATHAN JAN, CULVER CITY, CA (US)

(72) Inventor: JONATHAN JAN, CULVER CITY, CA (US)

(21) Appl. No.: 15/273,362

(22) Filed: Sep. 22, 2016

Related U.S. Application Data
(60) Provisional application No. 62/358,434, filed on Jul. 5, 2016.

(51) Int. Cl. A61F 13/02; A61F 13/00; A61L 15/22; A61L 15/44; A61L 15/28; A61L 15/42; A61L 15/58

(52) U.S. Cl. CPC A61F 13/0233; A61L 15/425; A61F 13/00063; A61F 13/0206; A61L 15/58; A61L 15/44; A61F 13/00012; A61L 15/28; A61L 15/22; A61L 2300/404; A61L 2300/606; A61L 2400/04

(57) ABSTRACT

A bandage to control bleeding from an open wound has a hemostatic gauze. An absorbent pad is removably attached to the hemostatic gauze.

### RELATED APPLICATIONS

This application claims priority to U.S. Provisional Application, 62/358,434 filed on Jul. 5, 2016, in the name of the same inventor as the present application, and which is incorporated by reference into the present application.

### TECHNICAL FIELD

The present application generally relates to a bandage, and, more particularly, to a bandage having chitosan material to aid in controlling bleeding from an open wound.

### BACKGROUND

Adhesive bandages are generally used to dress and protect relatively small or minor skin wounds and blemishes. Adhesive bandages typically comprise an elongated patch or strip (hereinafter strip). The strip generally has an adhesive element which allows the strip to be attached to the skin of the injured person. A gauze pad or similar item may be attached to the strip.

These known adhesive bandages, are commonly applied with slight pressure so that the gauze pad covers the wounded area. The gauze pad may aid in stopping the bleeding, as well as in protecting the wound from contamination. The gauze pad may further permit air flow in order to promote healing.

While these adhesive bandages work reasonably well, they have certain limitations. For example, while gauze pads do allow air to flow around the minor skin wounds, the gauze pads may keep the wound too moist if the wound has excessive drainage. If the wound remains too moist, infection may occur.

Further, adhesive bandages are generally used only for minor skin wounds and blemishes. These types of bandages are not used for larger wounds since the gauze pads are generally unable to stop/control the bleeding associated with larger wounds. Uncontrollable bleeding is a major cause of fatality. Stopping the bleeding is an ultimate challenge in emergency medical situations as well as surgeries. Knitted cellulosic fibers were adapted in the early 2000's to replace cotton gauze as the knitted cellulosic fibers are generally more effective in bleeding control. Later, potassium-phosphate powder was added to the gauze to form “gel” when reacting with blood plasma to aid in stopping bleeding.

Therefore, it would be desirable to provide an apparatus and method that overcome the above problems.

### SUMMARY

In accordance with one embodiment, a bandage to control bleeding from an open wound is disclosed. The bandage has a hemostatic gauze. An absorbent pad is removably attached to the hemostatic gauze.

In accordance with one embodiment, a bandage to control bleeding from an open wound is disclosed. The bandage has a non-woven chitosan fabric. An absorbent pad is removably attached to the non-woven chitosan fabric. A strip of material is provided, wherein an interior side of the strip of material has an adhesive material formed thereon. The non-woven chitosan fabric is attached to the interior side of the strip of material.

In accordance with one embodiment, a bandage to control bleeding from an open wound is disclosed. The bandage has a non-woven chitosan fabric which adheres and seals the open wound. An absorbent pad is removably attached to the non-woven chitosan fabric. A water soluble adhesive is applied to one of the non-woven chitosan fabric or the absorbent pad to removably attach the non-woven chitosan fabric to the absorbent pad. A porous-polymer coating is applied on the absorbent pad. A strip of material is provided, wherein an interior side of the strip of material has an adhesive material formed thereon, the non-woven chitosan fabric attached to the interior side of the strip of material.

### BRIEF DESCRIPTION OF THE DRAWINGS

The present application is further detailed with respect to the following drawings. These figures are not intended to limit the scope of the present application but rather illustrate certain attributes thereof.

FIG. 1 is an exploded perspective view of a bandage in accordance with one aspect of the present application;

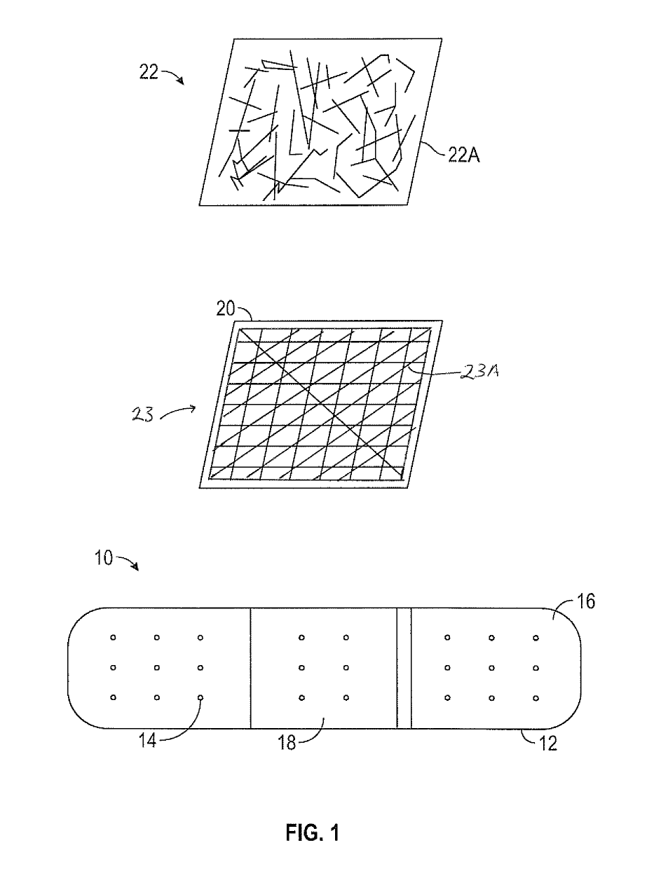

FIG. 2 is a magnified view of a knitted cellulosic gauze used in the bandage of FIG. 1 in accordance with one aspect of the present application; and

FIG. 3 is a magnified image of a non-woven chitosan fabric used in the bandage of FIG. 1 in accordance with one aspect of the present application.

### DESCRIPTION OF THE APPLICATION

The description set forth below in connection with the appended drawings is intended as a description of presently preferred embodiments of the disclosure and is not intended to represent the only forms in which the present disclosure may be constructed and/or utilized. The description sets forth the functions and the sequence of steps for constructing and operating the disclosure in connection with the illustrated embodiments. It is to be understood, however, that the same or equivalent functions and sequences may be accomplished by different embodiments that are also intended to be encompassed within the spirit and scope of this disclosure.

The present disclosure relates to an adhesive bandage that uses a plurality of fabrics for the purpose of stop bleeding effectively. The adhesive bandage may use an anti-bacterial fast-acting hemostatic gauze to stop bleeding effectively.

Referring to the FIGs., a bandage 10 may be seen. The bandage 10 may have a flexible strip of material 12 (strip 12). The strip 12 may be formed of a woven fabric, plastic (PVC, polyethylene or polyurethane), latex, or similar materials. The above is given as an example and should not be seen in a limiting manner. An interior side 16 of the strip 12 may have an adhesive material 14 formed thereon. The adhesive material 14 may be used to adhere the bandage 10 to the skin of the injured individual.

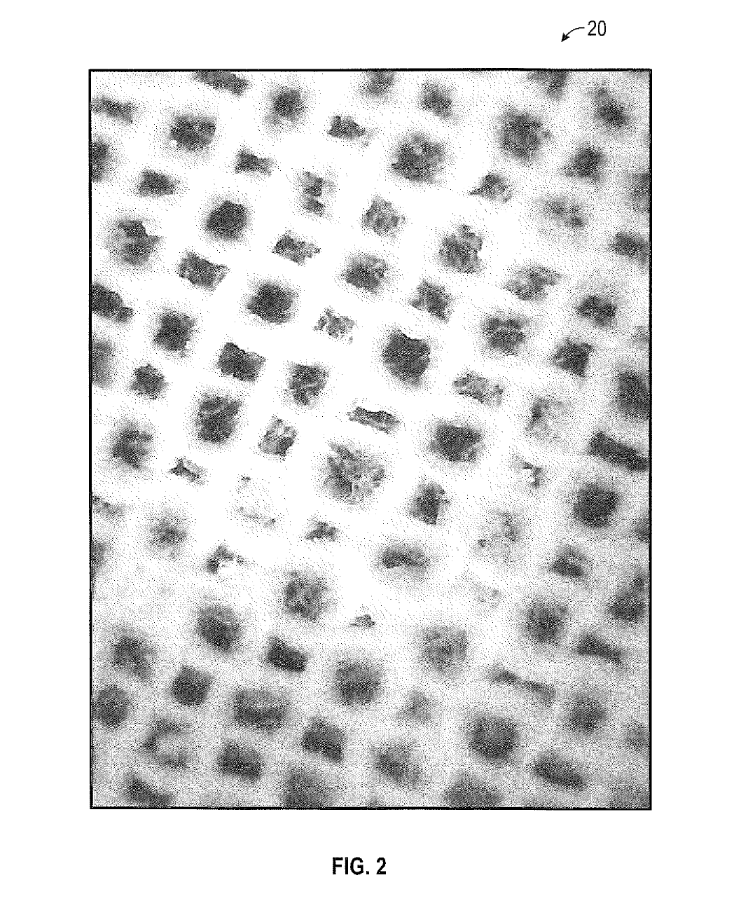

Located in a central area 18 of the interior side 16 of the strip 12 may be an absorbent pad 20. The absorbent pad 20 may be secured to the strip 12 through the adhesive material 14. The absorbent pad 20 may be made of cotton, linen, silk or similar material. As shown in FIG. 2, a microscopic image of the absorbent pad 20 may be seen. In this embodiment, the absorbent pad 20 may be formed of a non-woven material. For example, a non-woven cellulosic knitted fabric may be used. This type of architecture may be very porous and permeable.

A thin, porous-polymer coating may be applied over the absorbent pad 10. The polymer coating may be used to keep the absorbent pad 20 from sticking to the wound. The absorbent pad 20 may also be coated with an antiseptic solution. The antiseptic solution may be used to prevent the injured are from becoming infected.

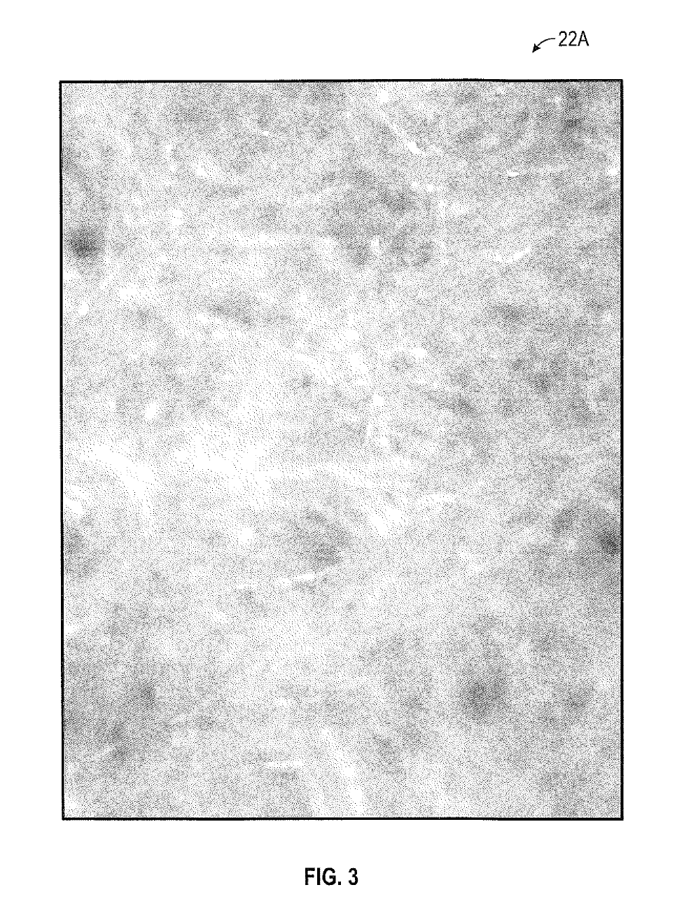

A hemostatic gauze 22 may be attached to the absorbent pad 20. The hemostatic gauze 22 may be uses to aid in stopping bleeding in the injured area where the bandage 10 may be applied. In accordance with one embodiment, the hemostatic gauze 22 may be a non-woven chitosan fabric 22A. As may be seen in FIG. 3, the architecture of the non-woven chitosan fabric 22A provides a much larger surface area for reaction with red blood cells. This may aid in blood clot formation. Chitosan is a positively charged polysaccharide that attracts blood cells which are negatively charged. This attraction causes an extreme adherence when in contact with blood. The red blood cells form a very tight coherent seal over the wound as they are drawn into the non-woven chitosan fabric 22A of the bandage 10. In accordance with one embodiment, the non-woven chitosan fabric 22A is not water soluble. This may allow blood plasma to pass through and be absorbed by the absorbent pad 20, while the chitosan reacts with red blood cells and forms a clot. Chitosan is also a proven natural anti-bacterial substance. This offers the added advantage of preventing secondary infections in the wounded area.

In accordance with one embodiment, the non-woven chitosan fabric 22A may be removably attached to the absorbent pad 20. The non-woven chitosan fabric 22A may be removably attached to the absorbent pad 20 in different manners. For example, the non-woven chitosan fabric 22A may be heat pressed to the absorbent pad 20. The heat press may form a bond between intersecting surfaces of the non-woven chitosan fabric 22A and the absorbent pad 20. Alternatively, the fibers of the non-woven chitosan fabric 22A and the absorbent pad 20 may be intertwined. For example, the fibers around the outer perimeter of the non-woven chitosan fabric 22A and the absorbent pad 20 may be intertwined such that the two may still be pulled apart. A needling machine may be used to intertwined the fibers of the non-woven chitosan fabric 22A and the absorbent pad 20. An adhesive material 23 may also be used to removably attach the non-woven chitosan fabric 22A and the absorbent pad 20 together.

In accordance with one embodiment, a water soluble adhesive 23A may be applied to one or more surfaces of the non-woven chitosan fabric 22A and/or the absorbent pad 20 to couple the non-woven chitosan fabric 22A to the absorbent pad 20. The water soluble adhesive 23A may be a spray type water soluble adhesive. A spray type water soluble adhesive may allow one to control a thinness of the adhesive material applied as opposed to one applied through a brushing application. The above are given as examples and should not be seen in a limiting manner. Other methods may be used to removably attach the non-woven chitosan fabric 22A and the absorbent pad 20 together.

In use, the bandage 10 is placed over the wounded area so that the non-woven chitosan fabric 22A engages with the open wound. The positively charged polysaccharide in the non-woven chitosan fabric 22A attracts blood cells which are negatively charged. This attraction causes an extreme adherence when in contact with blood. The red blood cells form a very tight coherent seal over the wound as they are drawn into the non-woven chitosan fabric 22A of the bandage 10. Since the non-woven chitosan fabric 22A is not water soluble, this may allow the blood plasma to pass through and be absorbed by the absorbent pad 20, while the chitosan reacts with red blood cells and forms the clot. The absorbent pad 20 may also be used to soak-up excessive blood. Since the non-woven chitosan fabric 22A is detachable from the absorbent pad 20, the bandage 10 may be removed from the skin of the individual. As the absorbent pad 20 absorbs the blood plasma, the water soluble adhesive 23A may dissolve and the absorbent pad 20 may be separated from the the non-woven chitosan fabric 22A leaving the non-woven chitosan fabric 22A relatively dry. Upon sealing and drying of the bleeding area, the non-woven chitosan fabric 22A, which is bio-degradable, may become an integral part of the seal.

The bandage 10 provides for an effective and fast formation of blood clot to stop bleeding of an open wound. Upon sealing and drying of the bleeding area, the woven chitosan fabric 22A, which is bio-degradable, becomes an integral part for sealing the open wound. The bandage 10 provides for a detachable bond between the non-woven chitosan fabric 22A and absorbent pad 20. The detachable bond between the non-woven chitosan fabric 22A and absorbent pad 20 allows for the bandage 10 to be easily removed. With the seal formed by the non-woven chitosan fabric 22A, the injured party may wash or shower without the risk of secondary bleeding.

While the bandage 10 has been described using the strip 12, in accordance with one embodiment, the bandage 10 may be formed without the strip 12. In this embodiment, the bandage 10 may have just the hemostatic gauze 22 attached to the absorbent pad 20. As in the previous embodiments, the hemostatic gauze 22 may be a non-woven chitosan fabric 22A. The non-woven chitosan fabric 22A may be removably attached to the absorbent pad 20. In use, the bandage 10 is placed over the wounded area so that the non-woven chitosan fabric 22A engages with the open wound. The positively charged polysaccharide in the non-woven chitosan fabric 22A attracts blood cells which are negatively charged. This attraction causes an extreme adherence when in contact with blood. The red blood cells form a very tight coherent seal over the wound as they are drawn into the non-woven chitosan fabric 22A of the bandage 10. The absorbent pad 20 may be used to soak-up excessive blood. Since the non-woven chitosan fabric 22A is detachable from the absorbent pad 20, the absorbent pad 20 may be removed from the skin of the individual. Upon sealing and drying of the bleeding area, the non-woven chitosan fabric 22A, which is bio-degradable, may become an integral part of the seal.

While embodiments of the disclosure have been described in terms of various specific embodiments, those skilled in the art will recognize that the embodiments of the disclosure may be practiced with modifications within the spirit and scope of the claims

1. A bandage to control bleeding from an open wound comprising:
a hemostatic gauze; and
an absorbent pad removably attached to the hemostatic gauze.

2. The bandage of claim 1, comprising a water soluble adhesive applied to one of the hemostatic gauze or the absorbent pad to removably attach the hemostatic gauze to the absorbent pad.

3. The bandage of claim 1, wherein the hemostatic gauze is heat pressed to the absorbent pad to removably attach the hemostatic gauze to the absorbent pad.

4. The bandage of claim 1, wherein fibers of the hemostatic gauze are intertwined with fibers of the absorbent pad to removably attach the hemostatic gauze to the absorbent pad.

5. The bandage of claim 1, wherein the hemostatic gauze is a non-woven chitosan fabric.

6. The bandage of claim 1, comprising a strip of material, wherein an interior side of the strip of material has an adhesive material formed thereon, the hemostatic gauze attached to the interior side of the strip of material.

7. The bandage of claim 1, comprising a porous-polymer coating applied on the absorbent pad.

8. The bandage of claim 1, comprising an antiseptic solution applied on the absorbent pad.

9. The bandage of claim 5, wherein the non-woven chitosan fabric is a non-woven, non-water soluable chitosan fabric which allows blood plasma to pass through and be absorbed by the absorbent pad, while the non-woven, non-water soluable chitosan fabric reacts with red blood cells to form a clot.

10. A bandage to control bleeding from an open wound comprising:
a non-woven chitosan fabric;
an absorbent pad removably attached to the non-woven chitosan fabric; and
a strip of material, wherein an interior side of the strip of material has an adhesive material formed thereon, the non-woven chitosan fabric attached to the interior side of the strip of material.

11. The bandage of claim 10, comprising a water soluble adhesive applied to one of the non-woven chitosan fabric or the absorbent pad to removably attach the non-woven chitosan fabric to the absorbent pad.

12. The bandage of claim 11, wherein the water soluble adhesive dissolves as the absorbent pad absorbs blood plasma.

13. The bandage of claim 10, wherein the non-woven chitosan fabric have an anti-bacterial substance preventing secondary infections.

14. The bandage of claim 10, comprising a porous-polymer coating applied on the absorbent pad.

15. The bandage of claim 10, comprising an antiseptic solution applied on the absorbent pad.

16. A bandage to control bleeding from an open wound comprising:
a non-woven chitosan fabric adhering and sealing the open wound;
an absorbent pad removably attached to the non-woven chitosan fabric;
a water soluble adhesive applied to one of the non-woven chitosan fabric or the absorbent pad to removably attach the non-woven chitosan fabric to the absorbent pad;
a porous-polymer coating applied on the absorbent pad; and
a strip of material, wherein an interior side of the strip of material has an adhesive material formed thereon, the non-woven chitosan fabric attached to the interior side of the strip of material.

16. The bandage of claim 16, comprising an antiseptic solution applied on the absorbent pad.

17. The bandage of claim 16, wherein the non-woven chitosan fabric is biodegradable sealing the open wound.

18. The bandage of claim 16, wherein the non-woven chitosan fabric is a non-woven, non-water soluable chitosan fabric allowing blood plasma to pass through and be absorbed by the absorbent pad, while the non-woven, non-water soluable chitosan fabric reacts with red blood cells to form a clot.

19. The bandage of claim 16, wherein the water soluble adhesive dissolves as the absorbent pad absorbs blood plasma.

20. The bandage of claim 16, wherein the non-woven chitosan fabric have an anti-bacterial substance preventing secondary infections.
